(19) United States
(12) Patent Application Publication
(10) Pub. No.: US 2026/0232892 A1
(43) Pub. Date: Aug. 13, 2026

(54) SYSTEM FOR REGULATING THE CONCENTRATION OF GLUCOSE IN THE BLOOD OF A PERSON

(71) Applicant: INREDA DIABETIC B.V., Goor (NL)

(72) Inventor: Robin KOOPS, Goor (NL)

(73) Assignee: INREDA DIABETIC B.V., Goor (NL)

(21) Appl. No.: 19/156,742

(22) PCT Filed: Feb. 21, 2024
(86) PCT No.: PCT/EP2024/054378

§ 371 Date: Aug. 14, 2025

(30) Foreign Application Priority Data
Feb. 21, 2023 (NL) 2034199

(51) Int. Cl. A61M 5/142; A61M 5/14; A61M 5/168

(52) U.S. Cl. CPC A61M 5/142; A61M 5/1407; A61M 5/16877; A61M 2005/14208; A61M 2202/0007; A61M 2202/04; A61M 2205/3303; A61M 2205/3327; A61M 2205/3334; A61M 2205/3379; A61M 2205/50; A61M 2230/005; A61M 2230/201

(57) ABSTRACT

A system for regulating the concentration of glucose in the blood of a person, comprising: a device for selectively supplying insulin and glucagon; at least two infusion sets; at least one sensor for measuring a glucose concentration in the blood of said person; and a controller for controlling said device such that a certain amount of insulin and glucagon is supplied per time unit, wherein said certain amount is chosen in accordance with the measured glucose concentration as prescribed by an insulin injection curve and a glucagon injection curve, wherein said controller is further arranged to determine if glucagon is supplied to said person in a chosen time frame, and wherein the controller is arranged to re-assess and optionally adjust the insulin injection curve based on the determination if glucagon is supplied to said person in said chosen time frame.

The invention relates to a system for regulating the concentration of glucose in the blood of a person, comprising:
a device for selectively supplying insulin and glucagon,
at least two infusion sets connectable to said device for infusing the insulin and glucagon to said person;
at least one sensor for measuring a glucose concentration in the blood of said person, and
a controller for controlling said device such that a certain amount of insulin and glucagon is supplied per time unit and thereby infused into the body of the person via a respective infusion set, wherein said certain amount is chosen in accordance with the measured glucose concentration as prescribed by an insulin injection curve and a glucagon injection curve which prescribes the amount of insulin and glucagon, respectively, to be supplied per measured glucose concentration.

Such a system is known from for example the international patent application with number WO2007/049961 in the name of the same applicant. WO2007/049961 in particular teaches a program which incorporates an adjustable glucagon injection curve represented by a first formula (ml(E)/fall mmol/l/unit of time) and an adjustable insulin injection curve represented by a second formula (ml(E)/rise mmol/l/unit of time) corresponding to an amount of glucagon or insulin respectively introduced into the body of the patient, and the nominal concentration of the glucose in the blood of the patient as a reaction thereto.

It is an object of the invention to improve the system described in WO2007/049961 and in particular the program thereof.

This object is achieved by providing a system according to the preamble, wherein said controller is further arranged to determine if glucagon is supplied to said person in a chosen time frame, and wherein the controller is arranged to re-assess and optionally adjust the insulin injection curve based on the determination if glucagon is supplied to said person in said chosen time frame.

When insulin is supplied in accordance with the measured glucose concentration as prescribed by the insulin injection curve as explained above, the glucose concentration in the blood of the person will decrease. However, if the glucose concentration in the blood decreases too much and comes below a certain threshold value, glucagon will be supplied in accordance with the measured glucose concentration as prescribed by the glucagon injection curve as explained above. When glucagon is supplied, the glucose concentration in the blood of the person will increase and when the glucose concentration increases too much insulin may again be supplied. Insulin and glucagon are thus supplied in accordance with their respective curves in such a manner as to try to maintain the glucose concentration in the blood within an agreeable band width or range. As will be clear from this explanation, there is a tendency to increase the supply of insulin after the supply of glucagon, because of the increasing glucose concentration resulting from the glucagon supply. However, the applicant has found that the supply of glucagon may also be an indication that too much insulin has been supplied, which has resulted in the glucose concentration becoming too low, such that glucagon had to be supplied. Therefore, the applicant has found that in case glucagon has been supplied, the insulin injection curve is preferably re-assessed and optionally adjusted. Therefore, in accordance with the present invention, the controller is arranged to determine if glucagon is supplied to said person in said chosen time frame, and to re-assess and optionally adjust the insulin injection curve based on the determination if glucagon is supplied to said person.

The sensor may be any suitable sensor, in particular any suitable glucose sensor. Said sensor may for example be a so called Continuous Glucose Monitoring (CMG) sensor for substantially continuously measuring the glucose concentration. Such sensor may for example measure the glucose concentration every few seconds, such as every 5, 10, 15, 20, 25, 30, 40, 45, 50, or 55 seconds, or every few minutes, such as every 1, 2, 3, 4, 5 minutes. It is preferred to measure the glucose concentration at least every 5 or 10 seconds.

The controller may be any suitable controller, for example a (micro)processor.

The controller may receive the measured glucose concentration from the sensor, wherein the sensor is able to communicate with the controller. Communication preferably takes place wirelessly, for example via a transmitter, but may alternatively take place via a wire.

Said glucagon injection curve may for example be the adjustable glucagon injection curve as represented by said first formula (ml(E)/fall mmol/l/unit of time) and the insulin injection curve may for example be the adjustable insulin injection curve represented by the second formula (ml(E)/rise mmol/l/unit of time) corresponding to the amount of glucagon or insulin respectively introduced into the body of the patient, and the nominal concentration of the glucose in the blood of the patient as a reaction thereto.

In an embodiment of the system according to the invention said chose time frame is a first chosen time frame, and wherein the controller is arranged to decrease the insulin injection curve if in said first chosen time frame the glucose concentration is below a first threshold value and if it is determined that glucagon is supplied at that time.

If the glucose concentration is below said first threshold at some time in said first chosen time frame and glucagon is supplied at that time, this may be an indication that too much insulin has been supplied and as such the controller is arranged to decrease the insulin injection curve.

In an embodiment of the system according to the invention said chose time frame is a first chosen time frame, and wherein the controller is arranged to increase the insulin injection curve if the average glucose concentration in said first chosen time frame is above a second threshold value and if it is determined that no glucagon is supplied in the chosen time frame. If the average glucose concentration is above said second threshold and no glucagon is supplied, this may be an indication that too little insulin has been supplied and as such the controller is arranged to increase the insulin injection curve.

In an embodiment of the system according to the invention said chose time frame is a first chosen time frame, and wherein said controller is arranged for controlling said device such that an additional amount of insulin and glucagon may be supplied dependent on the measured glucose concentration or on a slope of the measured glucose concentration, and wherein the controller is arranged to adjust the insulin injection curve if it is determined that an additional amount of glucagon is supplied in said first chosen time frame.

Such an additional amount of insulin or glucagon is in particular supplied in addition to an amount supplied as prescribed by the insulin and glucagon injection curve, respectively. Such an additional amount is supplied at glucose concentrations exceeding a certain threshold or if the slope of the measured glucose concentration exceeds a certain threshold. The slope of the measured glucose concentration is defined by the change of measured glucose concentration in time, i.e. the fall or rise of the measured glucose concentration in time. In case the glucose concentration is below a lower threshold for the glucose concentration or if the slope is a negative slope below a lower threshold for the glucose concentration slope, glucagon is supplied as said additional amount of glucagon. Such an additional amount of glucagon supplied may be an indication that too much insulin has been supplied as an amount prescribed by the insulin injection curve and the controller is arranged to adjust the insulin injection curve and in particular decrease the insulin injection curve.

The first chosen time frame in the above described embodiments may be a so-called examination time frame, an may for example a time frame having a duration of between two and four hours, preferably about three hours.

In an embodiment of the system according to the invention said chose time frame is a second chosen time frame, and wherein said controller is further arranged to determine the amount of glucagon supplied in said second chosen time frame, and wherein the controller is arranged to adjust the insulin injection curve if it is determined that the amount of glucagon supplied in said second chosen time frame exceeds a third threshold.

If the amount of glucagon supplied exceeds said third threshold, independent on the average glucose concentration in said chosen time frame, this may indicate that too much insulin has been supplied an the insulin injection curve is adjusted, in particular decreased.

Said second chosen time frame may be different from the first chosen time frame and may in particular be a longer time frame. For example said second chosen time frame may be around twenty four hours.

It is noted that the various thresholds and time frames are referred to as first, second, etc, just to be able to differentiate between the various thresholds and time frames and this is not intended to be limited in any way. For example, the second chosen time frame may be the only chosen time frame.

If it is described above and below that the insulin injection curve is adjusted, said insulin injection curve is adjusted by taking a percentage of the current curve, said percentage being chosen below 100% if the insulin injection curve is to be decreased and said percentage being chosen above 100% if the insulin injection curve is to be increased.

The invention will be further explained with respect to figures in which:

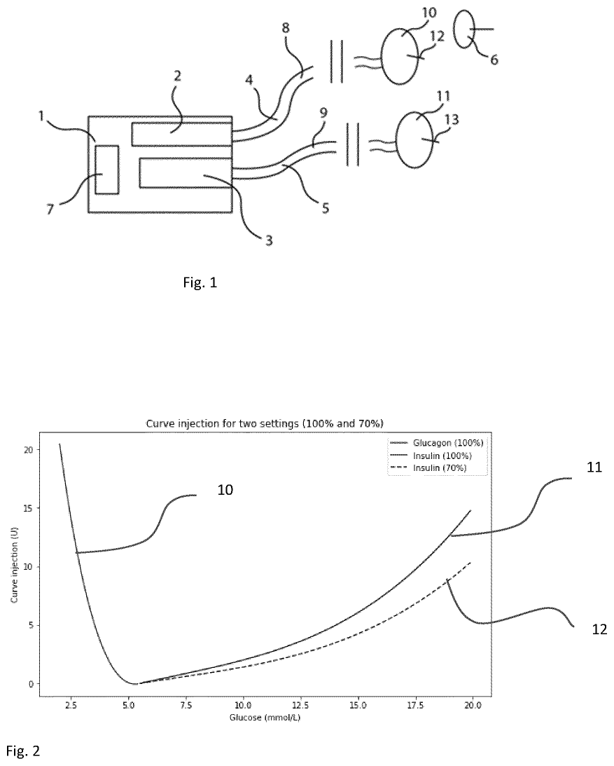

FIG. 1 schematically shows the system according to an embodiment of the invention;

FIG. 2 shows the curve injection for insulin and glucagon, wherein the curve for insulin may be adjusted in accordance with the current invention.

FIG. 1 shows a system for regulating the concentration of glucose in the blood of a person.

The system comprises in this exemplary embodiment a device 1 for selectively supplying two substances contained in respective reservoirs 2, 3, which reservoirs 2, 3 can be received in respective receiving spaces of the device 1. The two substances are insulin and glucagon, respectively. Respective two infusion sets 4, 5 are connected to said device 1 in a fluid communicating manner with the respective reservoirs 2, 3 for infusing a said substance to said person. In this embodiment the infusion sets 4,5 each comprise a respective hose or tube 8,9 for transporting a respective substance to an infusion element 10, 11 of that infusion set 4,5. The infusion elements 10, 11 each comprise a canula 12, 13 which may be inserted into the body of the person for infusing the substance into the body of the person. In this embodiment one sensor 6 for measuring a glucose concentration in the blood of said person is provided. In this embodiment the sensor 6 is shown to be a separate sensor but alternatively the sensor may be combined with one of the infusion sets 4, 5, in particular with the infusion element 10, 11 thereof. It may be preferred to provide two glucose sensors 6, wherein optionally each sensor 6 is combined with a respective infusion set. A controller 7 is provided, in this embodiment as part of the device 1, for controlling said device 1 such that a certain amount insulin and glucagon is supplied per time unit and thereby infused into the body of the person via the respective infusion set 4, 5, wherein said certain amount is chosen in accordance with the measured glucose concentration as prescribed by an insulin injection curve and a glucagon injection curve which prescribes the amount of insulin and glucagon, respectively, to be supplied per measured glucose concentration.

The insulin injection curve 11 and glucagon injection curve 10 are shown in FIG. 2 and prescribe the amount, in units, of insulin and glucagon to be injected in to the blood of the person in dependence on the measured glucose concentration in the blood of said person in mmol/L. In accordance with the present invention, the controller 7 of the device 1 is arranged to determine if glucagon is supplied to said person in a chosen time frame, and wherein the controller is arranged to re-assess and optionally adjust the insulin injection curve 11 based on the determination if glucagon is supplied to said person in said chosen time frame. Adjusting the insulin injection curve implies either increasing or decreasing the insulin injection curve, wherein the insulin injection curve is in particular adjusted by taking a percentage of the current insulin injection curve. In FIG. 1 the current insulin injection curve 11 is shown to be at 100% and an adjusted curve 12 is shown, which adjusted curve 12 70% of the insulin injection curve 11.

The insulin injection curve may in particular be adjusted in any one or more of the following situations:
1) if in said chosen time frame the glucose concentration is below a first threshold value and if it is determined that glucagon is supplied at that time, wherein said chosen time frame is a first chosen time frame;
2) the average glucose concentration in said chosen time frame is above a second threshold value and if it is determined that no glucagon is supplied in the chosen time frame, wherein said chosen time frame is said first chosen time frame;
3) if an additional amount of glucagon is supplied in said chosen time frame, wherein said chosen time frame is said first chosen time frame;
4) if the amount of glucagon supplied in said chosen time frame exceeds a third threshold value, wherein said chosen time frame is a second chosen time frame.

The first, third and fourth situations may in particular indicate that too much insulin has been supplied, which may have led to the supply of glucagon, and in such case the insulin injection curve may be decreased. The second situation may indicate that too little insulin may have been supplied, resulting in a glucose concentration that on average is too high in said time frame and to no glucagon supply, and in such case the insulin injection curve may be increased.

Said first chosen time frame may in particular be a so-called examination time frame, which may for example be a time of between 2 and 4 hours, more in particular for example about three hours. The examination time frame may start at any suitable moment. The second chosen time frame may be a different time frame and may for example be a time frame of 24 hours.

It is noted that the invention is not limited to the shown embodiments but also extends to variants within the scope of the appended claims.

1. A system for regulating the concentration of glucose in the blood of a person, comprising:
a device for selectively supplying insulin and glucagon;
at least two infusion sets connectable to said device for infusing the insulin and glucagon to said person;
at least one sensor for measuring a glucose concentration in the blood of said person; and
a controller for controlling said device such that a certain amount of insulin and glucagon is supplied per time unit and thereby infused into the body of the person via a respective infusion set, wherein said certain amount is chosen in accordance with the measured glucose concentration as prescribed by an insulin injection curve and a glucagon injection curve which prescribes the amount of insulin and glucagon, respectively, to be supplied per measured glucose concentration,
wherein said controller is further arranged to determine if glucagon is supplied to said person in a chosen time frame, and wherein the controller is arranged to re-assess and optionally adjust the insulin injection curve based on the determination if glucagon is supplied to said person in said chosen time frame.

2. The system according to claim 1, wherein said chose time frame is a first chosen time frame, and wherein the controller is arranged to decrease the insulin injection curve if in said first chosen time frame the glucose concentration is below a first threshold value and if it is determined that glucagon is supplied at that time.

3. The system according to claim 1, wherein said chose time frame is a first chosen time frame, and wherein the controller is arranged to increase the insulin injection curve if the average glucose concentration in said first chosen time frame is above a second threshold value and if it is determined that no glucagon is supplied in the chosen time frame.

4. The system according to claim 1, wherein said chose time frame is a first chosen time frame, and wherein said controller is arranged for controlling said device such that an additional amount of insulin and glucagon may be supplied dependent on the measured glucose concentration or on a slope of the measured glucose concentration, and wherein the controller is arranged to adjust the insulin injection curve if it is determined that an additional amount of glucagon is supplied in said first chosen time frame.

5. The system according to claim 1, wherein said chose time frame is a second chosen time frame, and wherein said controller is further arranged to determine the amount of glucagon supplied in said second chosen time frame, and wherein the controller is arranged to adjust the insulin injection curve if it is determined that the amount of glucagon supplied in said second chosen time frame exceeds a third threshold.

6. The system according to claim 1, wherein if said insulin injection curve is adjusted said insulin injection curve is adjusted by taking a percentage of the current curve, said percentage being chosen below 100% if the insulin injection curve is to be decreased and said percentage being chosen above 100% if the insulin injection curve is to be increased.
